(19) United States
(12) Patent Application Publication
(10) Pub. No.: US 2023/0158012 A1
(43) Pub. Date: May 25, 2023

(54) ORAL LIQUID FORMULATIONS OF LENVATINIB

(71) Applicant: SHILPA MEDICARE LIMITED, RAICHUR (IN)

(72) Inventors: NIJAGUNI REVANSIDDAYYA RUDRASWAMY MATH, Bengaluru (IN); PRADEEP SHIVAKUMAR, Bengaluru (IN)

(73) Assignee: SHILPA MEDICARE LTD, RAICHUR (IN)

(21) Appl. No.: 17/922,978

(22) PCT Filed: May 13, 2021
(86) PCT No.: PCT/IB2021/054076

§ 371 Date: Nov. 3, 2022

(30) Foreign Application Priority Data
May 23, 2020 (IN) 201941053406

(51) Int. Cl. A61K 31/47; A61K 9/00; A61K 47/10; A61K 47/38

(52) U.S. Cl. CPC A61K 31/47; A61K 9/0053; A61K 47/10; A61K 47/38

(57) ABSTRACT

The present invention relates to oral liquid formulations comprising lenvatinib or pharmaceutically acceptable salts or solvates thereof with improved stability and palatability. Further the present invention relates to an oral pharmaceutical suspension comprising lenvatinib or pharmaceutically acceptable salt, solvate or hydrate thereof and pharmaceutically acceptable excipients, wherein pharmaceutically acceptable excipients are selected from suspending agents, wetting agents, stabilizing agents, vehicle, organic co-solvents, sweeteners, flavoring agents, preservatives, antioxidants and buffering agents.

### FIELD OF THE INVENTION

The present invention relates to an oral liquid pharmaceutical formulation comprising lenvatinib or a pharmaceutically acceptable salt, solvate or hydrate thereof and one or more pharmaceutically acceptable excipients.

### BACKGROUND OF THE INVENTION

E7080 (also known as lenvatinib mesylate) is an active inhibitor of multiple receptor tyrosine kinases (e.g., receptor tyrosine kinases involved in angiogenesis and tumor proliferation) including vascular endothelial growth factor (VEGF), fibroblast growth factor (FGF), platelet-derived growth factor receptor a (PDGFRa), KIT, and RET proto-oncogene receptors. Lenvatinib mesylate is structurally represented as:

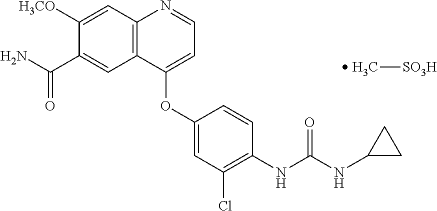

The drug with the name lenvatinib, contains lenvatinib mesylate, has been approved by the Food and Drug Administration (FDA) for the treatment of locally recurrent or metastatic, progressive thyroid cancer, resistant to radioactive iodine and for the treatment of advanced renal carcinoma.

U.S. Pat. No. 7,253,286 discloses the lenvatinib and its pharmaceutically acceptable salts such as hydrochloric acid salts, sulfuric acid salts, carbonic acid salts, bicarbonate salts, hydrobromic acid salts and hydroiodic acid salts; organic carboxylic acid addition salts such as acetic acid salts, maleic acid salts, lactic acid salts, tartaric acid salts, trifluoroacetic acid salts, methanesulfonic acid salts, hydroxymethanesulfonic acid salts, hydroxyethanesulfonic acid salts, benzenesulfonic acid salts, toluenesulfonic acid salts, taurine salts, trimethylamine salts, triethylamine salts, pyridine salts, procaine salts, picoline salts, dicyclohexyl amine salts, N,N′-dibenzyl ethyl enediamine salts, N-methylglucamine salts, diethanolamine salts, triethanolamine salts, tris(hydroxymethylamino)methane salts, phenethylbenzyl amine salts, arginine salts, lysine salts, serine salts, glycine salts, aspartic acid salts and glutamic acid salts.

US Publication No. 20080214557A1 discloses a method for preparing the pharmaceutical composition comprising blending, in a pharmaceutical composition containing a pharmaceutically active ingredient (lenvatinib mesylate), at least one disintegrant and at least one water-soluble salt having a pH of from 3 to 9 in an aqueous solution of 2.5% concentration. The water-soluble salt used in US '557 Publication are selected from group consisting of sodium chloride, magnesium chloride, sodium bicarbonate, potassium chloride and ammonium chloride. Further US '557 Publication discloses the tablet dosage forms that employ a water-soluble salt, especially a water-soluble inorganic salt commonly used as a drug additive, such as sodium chloride or potassium chloride, together with a disintegrant such as low-substituted hydroxypropyl cellulose, the disintegrability of pharmaceutical preparations can be markedly improved.

U.S. Pat. No. 8,969,379 discloses the pharmaceutical composition comprising lenvatinib or a salt thereof, (i) 1-10% w/w of one or more compounds selected from group consisting of magnesium oxide, sodium carbonate, disodium hydrogenphosphate, sodium citrate, dipotassium hydrogenphosphate, sodium acetate, sodium hydrogencarbonate and sodium hydroxide, and (ii) one or more compounds selected from the group consisting of light anhydrous silicic acid, silicon dioxide hydrate and calcium silicate. US Patent '379 discloses the stability evaluation of lenvatinib mesylate and combined with the magnesium oxide, sodium carbonate, disodium hydrogenphosphate, sodium citrate, dipotassium hydrogenphosphate, sodium acetate, sodium hydrogencarbonate, sodium hydroxide, glycine and Gluconolactone that exhibit the pH values of 10.63, 11.45, 9.26, 8.22, 9.11, 8.46, 8.15, 13.56, 6.17 and 2.40 respectively when 5% w/w aqueous solutions or suspensions were made and analyzed for the impurities of lenvatinib mesylate on storage. The results demonstrated that when the pH value of 5% (w/w) aqueous solution or suspension is more than 8 or more, the decomposition can be significantly reduced. Further US '379 patent discloses the tablet dosage forms that are dissolved in water to make 5% (w/w) aqueous solution or suspension.

US Publication No. 20180028662A1 discloses the method for suppressing the bitterness of lenvatinib or a pharmaceutically acceptable salts with the basic substance. The US Publication No. '662 further discloses the administration method of lenvatinib by
1. suspending in an aqueous solvent in a vessel, a pharmaceutical composition (granules) comprising lenvatinib and basic substance,
2. administering the suspension obtained in 1) from the vessel to a patient,
3. rinsing the vessel with an aqueous solvent, and
4. administering a rinsing solution obtained in 3) to the patient employed.

The liquid dosage form of lenvatinib administered to patients as disclosed in US Publication No. '662, '557 and US Patent No. '379 is prepared by suspending the granules or tablets in a solvent which requires the intervention of the patients to prepare the suspension or solution for administration.

In order to overcome the above disadvantages, there exists a need to develop an oral liquid dosage forms of lenvatinib or pharmaceutically acceptable salts or solvates thereof which are easily administered to patients without preparation of suspension from the granules or tablets. Therefore, the inventors of the present invention have developed an oral liquid dosage forms of lenvatinib or pharmaceutically acceptable salts or solvates thereof which masks the bitter taste of the lenvatinib or pharmaceutically acceptable salts or solvates thereof exhibiting improved stability and palatability.

### OBJECTS OF THE INVENTION

The main objective of the present invention is to provide an oral liquid formulation comprising lenvatinib or pharmaceutically acceptable salts or solvates thereof with improved stability and palatability.

Another object of the present invention is to provide oral solution comprising lenvatinib or pharmaceutically acceptable salts or solvates thereof with improved stability and palatability.

In yet another object, the present invention is to provide oral suspension comprising lenvatinib or pharmaceutically acceptable salts or solvates thereof with improved stability and palatability.

Another object of the present invention is to provide an oral suspension of lenvatinib or pharmaceutically acceptable salts or solvates thereof that exhibits similar bioavailability to commercial lenvatinib mesylate capsules (Lenvima®).

Another object of the present invention is to provide oral suspension of lenvatinib or pharmaceutically acceptable salts or solvates thereof having dose flexibility for patients who needs special doses of drug and have difficulties in swallowing capsule dosage forms.

### SUMMARY OF THE INVENTION

The present invention relates to an oral liquid formulation comprising lenvatinib or pharmaceutically acceptable salt, solvate or hydrate thereof and pharmaceutically acceptable excipients.

The present invention further relates to an oral pharmaceutical suspension comprising lenvatinib or pharmaceutically acceptable salt, solvate or hydrate thereof and pharmaceutically acceptable excipients, wherein pharmaceutically acceptable excipients are selected from suspending agents, wetting agents, stabilizing agents, vehicle, organic co-solvents, sweeteners, flavoring agents, preservatives, antioxidants and buffering agents.

### DETAILED DESCRIPTION OF THE INVENTION

The present invention provides an oral liquid formulation comprising lenvatinib or pharmaceutically acceptable salt, solvate or hydrate thereof and pharmaceutically acceptable excipients.

The present invention provides an oral an oral pharmaceutical suspension comprising lenvatinib or pharmaceutically acceptable salt, solvate or hydrate thereof and pharmaceutically acceptable excipients with improved stability and higher rate of bioavailability.

The present invention provides an oral pharmaceutical suspension comprising lenvatinib or pharmaceutically acceptable salt, solvate or hydrate thereof and pharmaceutically acceptable excipients, wherein pharmaceutically acceptable excipients are selected from suspending agents, wetting agents, stabilizing agents, vehicle, organic co-solvents, sweeteners, flavoring agents, preservatives, antioxidants and buffering agents.

In specific embodiment, the present invention provides an oral pharmaceutical suspension comprising lenvatinib mesylate and pharmaceutically acceptable excipients, wherein pharmaceutically acceptable excipients are selected from suspending agents, wetting agents, stabilizing agents, vehicle, organic co-solvents, sweeteners, flavoring agents, preservatives, antioxidants and buffering agents.

The lenvatinib mesylate is preferably lenvatinib mesylate with 95 to 99% of particles having an equivalent diameter less than about 40 microns. Even more preferably the lenvatinib mesylate particles having an equivalent diameter less than about 10 microns are obtained by micronization.

The amount of lenvatinib or pharmaceutically acceptable salt, solvate or hydrate thereof present in the suspension should be sufficient to provide a therapeutic amount of the active and a convenient dosage unit.

In one embodiment of the invention, the suspension comprises of about 0.5 mg/mL to about 50 mg/mL of lenvatinib mesylate, preferably about 1 mg/mL to about 40 mg/mL of lenvatinib mesylate, more preferably about 2 mg/mL to about 25 mg/mL of lenvatinib mesylate and most preferably about 4.9 mg/mL of lenvatinib mesylate. Accordingly, lenvatinib mesylate shall be present in an amount of from about 0.05% w/v to about 5.0% w/v of the suspension, preferably about 0.1% w/v to about 4.0% w/v of the suspension, more preferably about 0.2% w/v to about 2.5% w/v of suspension and most preferably of about 0.49% w/w of suspension.

Suspending agents preferably used in the lenvatinib mesylate suspension of the present invention are selected from carbomers, polyvinyl alcohol, polyvinyl pyrrolidone, hydroxypropylmethyl cellulose, hydroxyethyl cellulose, hydroxypropyl cellulose, methyl cellulose, carboxymethyl cellulose, microcrystalline cellulose, powdered cellulose, xanthan gum, gellan gum, carageenan, acacia, tragacanth, gelatin, guar gum, alginic acid, sodium alginates, propylene glycol alginate, eudragit (methacrylic acid and methyl methacrylate copolymer), dextrin, dextran, dextran-polyethylene glycol conjugates, and the glycosaminoglycans family of polymers, such as heparin sulfate, heparin sulfate, dermatan sulfate, chondroitin sulfate.

In one embodiment, the suspension of the present invention comprises of about 0.5 mg/mL to about 50 mg/mL of suspending agent, preferably about 1 mg/mL to about 40 mg/mL of suspending agent and most preferably about 2 mg/mL to about 20 mg/mL of suspending agent.

Accordingly suspending agent shall be present in an amount of from about 0.05% w/v to about 5.0% w/v of the suspension, preferably about 0.1% w/v to about 4.0% w/v of the suspension, and most preferably about 0.2% w/v to about 2.0% w/v of suspension.

The most preferably used suspending agent is mixture of microcrystalline cellulose and carboxymethyl cellulose, e.g., its sodium salt. Preferably the ratio of carboxymethyl cellulose to microcrystalline cellulose in the mixture is 1:5 to 1:12, e.g., 1:8 to 1:10. As a preferred mixture one may use dispersible cellulose, e.g. as known under the trade name Avicel® RC, e.g. Avicel® RC 591, commercially available from e.g. FMC Corporation USA. The mixture of carboxymethyl cellulose e.g., its sodium salt and microcrystalline cellulose is present in range of about 1 mg/mL to about 40 mg/mL, preferably in the range of about 2 mg/mL to about 20 mg/mL and most preferably 15 mg/mL. The suspension of this invention provides various advantages including an absence of “lumps” even after long storage when the composition is shaken for use, as well as a highly improved pourability. Moreover, such composition is stable e.g. at least 3 months, 6 months, 12 months, 18 months, 24 months and 36 months at controlled room temperature, and well tolerated for oral administration. Alternately, such compositions are stable e.g. at least 3 months, 6 months at 40° C./75% RH or at 40° C./25% RH.

Wetting agents preferably used in the lenvatinib mesylate suspension of present invention are selected from polyethylene glycol stearates, for example monostearate of polyethylene glycol 400, polaxamer and polysorbate. The most preferably used wetting agent is polyethylene glycol 400 monostearate.

In one embodiment, suspension of the present invention comprises about 0.05 mg/mL w/v to about 10 mg/mL, more preferably about 0.1 mg/mL to about 3 mg/mL of wetting agent. Accordingly wetting agent shall be present in an amount of from about 0.005% w/v to about 1.0% w/v of the suspension, preferably about 0.01% w/v to about 0.3% w/v of the suspension.

In a specific embodiment, suspension of the present invention comprises of about 0.05 mg/mL to about 10 mg/mL of polyethylene glycol monostearate, more preferably about 0.1 mg/mL to about 3 mg/mL of polyethylene glycol monostearate and most preferably 0.1 mg/mL of polyethylene glycol monostearate.

Stabilizing agents preferably used in the lenvatinib mesylate suspension of present invention are selected from calcium hydroxide and potassium hydroxide.

In one embodiment of the invention, suspension of present invention comprises of about 0.1 mg/mL to about 100 mg/mL of stabilizing agent, preferably of about 1 mg/mL to about 50 mg/mL of stabilizing and most preferably of about 2 mg/mL to about 40 mg/mL of stabilizing agent. Accordingly stabilizing agent shall be present in an amount of from about 0.01% w/v to about 10.0% w/v of the suspension, preferably about 0.1% w/v to about 5.0% w/v of the suspension, and most preferably about 0.2% w/v to about 4.0% w/v of suspension.

In specific embodiment, suspension of the present invention comprises of about 0.5 mg/mL to about 35 mg/mL of calcium hydroxide as a stabilizing agent, more preferably of about 1 mg/mL to about 25 mg/mL of calcium hydroxide as a stabilizing agent and most preferably of about 2 mg/mL to about 20 mg/mL of calcium hydroxide as a stabilizing agent. Accordingly, calcium hydroxide shall be present in an amount of from about 0.05% w/v to about 3.5% w/v of the suspension, preferably about 0.1% w/v to about 2.5% w/v of the suspension, and most preferably about 0.2% w/v to about 2.0% w/v of suspension.

In another specific embodiment, suspension of the present invention comprises of about 0.1 mg/mL to about 15 mg/mL of potassium hydroxide as a stabilizing agent, more preferably of about 0.5 mg/mL to about 10 mg/mL of potassium hydroxide as a stabilizing agent and most preferably of about 1 mg/mL to about 5 mg/mL of potassium hydroxide as a stabilizing agent. Accordingly, potassium hydroxide shall be present in an amount of from about 0.01% w/v to about 1.5% w/v of the suspension, preferably about 0.05% w/v to about 1.0% w/v of the suspension, and most preferably about 0.1% w/v to about 0.5% w/v of suspension.

Vehicles used in the present lenvatinib mesylate suspension are mainly liquid which carry lenvatinib mesylate and other excipients in dissolved or dispersed state. Pharmaceutical vehicles can be classified as aqueous vehicles and oily vehicles. Aqueous vehicles include purified water, alcoholic solvents selected from ethyl alcohol. Oily vehicles include vegetable oils, mineral oils, organic oily bases or emulsified bases. In the present invention purified water is used as the Vehicle.

Organic co-solvents used in the present lenvatinib mesylate suspension are selected from the group consisting of propylene glycol and polyethylene glycol.

In one embodiment, suspension of the present invention comprises of about 0.1 mg/mL to about 100 mg/mL of propylene glycol as a co-solvent, more preferably of about 0.5 mg/mL to about 50 mg/mL of propylene glycol as a co-solvent and most preferably of about 1 mg/mL to about 30 mg/mL of propylene glycol as a co-solvent. Accordingly, propylene glycol shall be present in an amount of from about 0.01% w/v to about 10.0% w/v of the suspension, preferably about 0.05% w/v to about 5.0% w/v of the suspension, and most preferably about 0.1% w/v to about 3.0% w/v of suspension.

Sweeteners are added in the liquid formulations to impart sweetness and improve patient compliance through taste masking. The main sweetener employed in oral preparations can be selected from but not limited to sucrose, liquid glucose, sorbitol, saccharin sodium, stevoside, saccharin sodium and aspartame.

Flavoring agents are added to increase patient acceptance of the drug by masking the specific taste sensations. Flavoring agent can be selected but not limited to essential oils including peppermint oil, orange oil, lemon oil or the fruit flavors like cherry flavour and banana flavour.

Preservatives are included in the pharmaceutical suspension to prevent the growth of the microorganisms during the product manufacturing and shelf-life. Preservatives can be selected from but not limited to propylene glycol, benzoic acid, potassium sorbate, sodium benzoate and chlorobutanol.

Antioxidants can reduce drug oxidation. Antioxidants can also act as chain terminators, reacting with free radicals in liquid to stop the free-radical propagation cycle. Oxidation may lead to products with an unpleasant odour, taste, appearance, precipitation, discoloration or even a slight loss of activity. The main antioxidants employed in oral preparations can be selected from but not limited to α-tocopherol acetate, ascorbic acid, butylated hydroxytoluene, butylated hydroxyanisole and sodium bisulfate.

Buffering agents used in the present invention are selected from the group consisting of acetate buffer (acetic acid), phosphate buffer (phosphoric acid), succinate buffer (succinic acid) and citrate buffer (citric acid), histidine, lactic acid, tromethamine, aspartic acid, glutamic acid, tartaric acid, malic acid, fumaric acid and gluconic acid. Lenvatinib mesylate suspension of the present invention has a pH of about 5.0 to about 14.0, more preferably pH of about 5.5 to about 12.0 and most preferably of about 5.8 to about 10.0.

A typical composition according to the invention is expressed as follows.

| S. No | Name of Ingredient | mg/mL | Formula % w/v |
|---|---|---|---|
| 1. | Lenvatinib Mesylate | 0.5-50 | 0.05-5.0 |
| 2. | Suspending agent | 0.5-50 | 0.05-5.0 |
| 3. | Wetting agent | 0.05-10 | 0.005-1.0 |
| 4. | Organic co-solvent | 0.1-100 | 0.01-10.0 |
| 5. | Sweetener | 0.01-50 | 0.1-5.0 |
| 6. | Stabilizing agent | 0.1-100 | 0.1-10.0 |
| 7. | Antioxidant | 0.01-2 | 0.001-0.2 |
| 8. | Flavouring agent | 0.01-2 | 0.001-0.2 |
| 9. | Buffering agent | To adjust pH 5.0 to | To adjust pH 5.0 to |
| | | 14.0 | 14.0 |
| 10. | Vehicle | q.s to 1 mL | q.s |

More specifically the typical composition according to the present invention is expressed as follows.

| S. No | Name of Ingredient | mg/mL | Formula % w/v |
|---|---|---|---|
| 1. | Lenvatinib Mesylate | 0.5-50 | 0.05-5.0 |
| 2. | Microcrystalline cellulose | 0.5-50 | 0.05-5.0 |
| | and | | |
| | Carboxymethyl cellulose | | |
| | (Avicel RC 591) | | |
| 3. | Polyethylene glycol 400 | 0.05-10 | 0.005-1.0 |
| | monostearate | | |
| 4. | Propylene glycol | 0.1-100 | 0.01-10.0 |
| 5. | Saccharin sodium | 0.01-1.0 | 0.001-0.1 |
| 6. | Sorbitol | 0.01-50 | 0.1-5.0 |
| 7. | Calcium hydroxide | 0.5-35 | 0.05-3.5 |
| 8. | Potassium hydroxide | 0.1-15 | 0.01-1.5 |
| 9. | Butylated hydroxy toluene | 0.01-2 | 0.001-0.2 |
| 10. | Cherry flavour | 0.01-2 | 0.001-0.2 |
| 11. | Citric acid | To adjust pH 5.0 | To adjust pH 5.0 |
| | | to 14.0 | to 14.0 |
| 12. | Vehicle | q.s to 1 mL | q.s |

The oral pharmaceutical suspension of the above composition is prepared by following steps irrespective to order of addition, but not limited to:
1. Add suspending agent, wetting agent, sweetener, stabilizing agent, antioxidant, flavouring agent and organic co-solvent in vehicle till it dissolves or disperses.
2. Add lenvatinib mesylate and mix till it dissolves or disperse.
3. Add the buffering agent to adjust the pH of from about 5.0 to about 14.0.
4. Makeup volume to the desired batch size.

The formulations of the invention are useful for the known indications of differentiated thyroid cancer, renal cell carcinoma, hepatocellular carcinoma and endometrial cancer.

The process of preparing an oral suspension may be carried out in an inert e.g. stainless-steel reactor vessel optionally under an inert atmosphere, e.g. nitrogen.

The resultant oral Suspension is preferably maintained under an inert atmosphere and is transferred to containers, bottles. In a further aspect the present invention relates to a container having a fill volume of, e.g., from about 50 ml to about 300 ml comprising a lenvatinib suspension as previously described. Containers may be chosen which are made of material which is non-reactive or substantially non-reactive with the oral suspension.

Containers for use in the storage of the oral suspensions according to the invention may be used to administer a multiple dose of active agent. The device used to convey the oral suspension from the container into the body of a patient may be any of the devices commonly used in the art to deliver therapeutic agents as oral Suspensions from containers, such as high- or low-volume containers. Preferably containers according to the present invention comprise a dosing syringe adapted to fit to said container.

The following examples are provided to illustrate the present invention. It is understood, however, that the invention is not limited to the specific conditions or details described in the examples below. The examples should not be construed as limiting the invention as the examples merely provide specific methodology useful in the understanding and practice of the invention and its various aspects. While certain preferred and alternative embodiments of the invention have been set forth for purposes of disclosing the invention, modification to the disclosed embodiments can occur to those who are skilled in the art.

### Example 1

Oral Suspension of Lenvatinib Mesylate (4 mg/mL of Lenvatinib)

| S. No | Ingredient | mg/mL | | (% w/v) |
|---|---|---|---|---|
| 1. | Lenvatinib mesylate | 4.9 | | 0.49% |
| 2. | Avicel RC 591 | 15 | | 1.5% |
| | (Microcrystalline cellulose & | | | |
| | Carboxymethyl cellulose) | | | |
| 3. | Polyethylene glycol 400 | 0.1 | | 0.01% |
| | monostearate | | | |
| 4. | Propylene glycol | 25 | | 2.5% |
| 5. | Saccharin sodium | 0.05 | | 0.005% |
| 6. | Sorbitol | 25 | mg | 2.5% |
| 7. | Calcium hydroxide | 14 | mg | 1.4% |
| 8. | Potassium hydroxide | 3 | mg | 0.3% |
| 9. | Butylated hydroxytoluene | 0.15 | mg | 0.015% |

| | | | |
|---|---|---|---|
| 10. | Phosphate buffer | Q.s to adjust pH 5-8 | Q.S |
| 11. | Purified water | Q.S | Q.S |

Process for Preparation:
1. Add Avicel RC 591, Polyethylene glycol 400 monostearate, propylene glycol, saccharin sodium, sorbitol, calcium hydroxide, potassium hydroxide, butylated hydroxytoluene to purified water one by one to dissolve or disperse.
2. Add lenvatinib mesylate to contents of step 1 and dissolve or disperse.
3. Add the phosphate buffer to adjust the pH of from about 5.0 to about 14.0.
4. Makeup volume to the desired batch size.

### Example—2

Oral Suspension of Lenvatinib Mesylate (4 mg/mL of Lenvatinib)

| S. No | Ingredient | mg/mL | (% w/v) |
|---|---|---|---|
| 1. | Lenvatinib mesylate | 4.9 | 0.49% |
| 2. | Avicel RC 591 | 150 | 1.5% |
| | (Microcrystalline cellulose & | | |
| | Carboxymethyl cellulose) | | |
| 3. | Polyethylene glycol 400 | 0.1 | 0.01% |
| | monostearate | | |
| 4. | Propylene glycol | 25 | 2.5% |
| 5. | Saccharin sodium | 3.34 | 0.33% |
| 6. | Sorbitol | 25 | 2.5% |
| 7. | Calcium hydroxide | 14 | 1.4% |
| 8. | Potassium hydroxide | 3 | 0.3% |
| 9. | Butylated hydroxytoluene | 0.15 | 0.015% |
| 10. | Citric acid anhydrous | 27 | 2.7% |
| 11. | Cherry Flavor | 0.4 | 0.04% |
| 12. | Purified water | Q.S | Q.S |

Process for Preparation
1. Add Avicel RC 591, Polyethylene glycol 400 monostearate, propylene glycol, saccharin sodium, sorbitol, calcium hydroxide, potassium hydroxide, butylated hydroxytoluene, cherry flavour to purified water one by one to dissolve or disperse.
2. Add lenvatinib mesylate to contents of step 1 and dissolve or disperse.
3. Add the citric acid to adjust the pH of from about 5.0 to about 14.0.
4. Makeup volume to the desired batch size and fill into desirable container.

We claim:

1. An oral liquid suspension formulation comprising
(a) about 0.5 mg/mL to about 50 mg/mL of lenvatinib mesylate and
(b) a pharmaceutically acceptable excipient.

2. The oral liquid suspension formulation as claimed in claim 1, wherein pharmaceutically acceptable excipients are selected from the group consisting of suspending agents, wetting agents, stabilizing agents, vehicle, organic co-solvents, sweeteners, flavoring agents, preservatives, antioxidants and buffering agents.

3. The oral liquid suspension formulation as claimed in claim 1, wherein the formulation comprises
(a) about 0.5 mg/mL to about 50 mg/mL suspending agent,
(b) about 0.05 mg/mL to about 10 mg/mL wetting agent,
(c) about 0.1 mg/mL to about 100 mg/mL organic co-solvent,
(d) about 0.1 mg/mL to about 100 mg/mL stabilizing agent,
(e) sweetener,
(f) antioxidant,
(g) flavoring agent,
(h) buffering agent and
(i) purified water, wherein the pH of the formulation is of about 5.0 to about 14.0.

4. The oral liquid suspension formulation as claimed in claim 3, wherein suspending agent is mixture of carboxymethyl cellulose and microcrystalline cellulose.

5. The oral liquid suspension formulation as claimed in claim 3, wherein wetting agent is selected from group consisting of polyethylene glycol stearate polaxamer and polysorbate.

6. The oral liquid suspension formulation as claimed in claim 3, wherein stabilizing agent is selected from group consisting of calcium hydroxide and potassium hydroxide.

7. The oral liquid suspension formulation as claimed in claim 3, wherein organic co-solvent is selected from group consisting of polyethylene glycol and propylene glycol.

8. An oral liquid suspension formulation comprising
(a) about 0.5 mg/mL to about 50 mg/mL of lenvatinib mesylate,
(b) a mixture of carboxymethyl cellulose and microcrystalline cellulose in an amount of about 1 mg/mL to about 40 mg/mL,
(c) about 1 mg/mL to about 25 mg/mL of calcium hydroxide, and
(d) about 0.5 mg/mL to about 10 mg/mL of potassium hydroxide.

9. The oral liquid suspension formulation as claimed in claim 1, having a pH of about 5.0 to about 14.0.

10. An oral liquid suspension formulation comprising
(a) about 0.5 mg/mL to about 50 mg/mL of lenvatinib mesylate,
(b) a mixture of carboxymethyl cellulose and microcrystalline cellulose in an amount of about 1 mg/mL to about 40 mg/mL,
(c) about 1 mg/mL to about 25 mg/mL of calcium hydroxide,
(d) about 0.5 mg/mL to about 10 mg/mL of potassium hydroxide,
(e) about 0.05 mg/mL to about 10 mg/mL of polyethylene glycol monostearate,
(f) about 1 mg/mL to about 30 mg/mL of propylene glycol,
(g) sweetener,
(h) antioxidant,
(i) buffering agent,
(j) flavouring agent and
(k) purified water, wherein the pH of the composition is of about 5.0 to about 14.0.
